(19) United States
(12) Plant Patent Application Publication
(10) Pub. No.: US 2009/0188012 P1
(43) Pub. Date: Jul. 23, 2009

(54) PAULOWNIA TREE NAMED 'ANAGENESIS TRIFOLIA'

(50) ;

(76) Inventor: Scot Corbett, Lenox, GA (US)

Correspondence Address: COATS & BENNETT, PLLC, 1400 Crescent Green, Suite 300, Cary, NC, 27518, US

(21) Appl. No.: 12/009,422

(22) Filed: Jan. 18, 2008

(51) Int. Cl. A01H 5/00

(52) U.S. Cl. PLT/216

(57) ABSTRACT

A new and distinct variety of Paulownia tree which combines the fast growing characteristics and drought tolerance of Paulownia Elongata, the straight growth characteristics of Paulownia Fortunei and the cold tolerance of Paulownia Catalpifolia. The Paulownia Anagenesis Trifolia is characterized by its flower color and its set of tri-pod branches evenly spaced around the tree trunk as they extend up the tree trunk at approximately 120 degree increments.

### LATIN NAME OF THE GENUS AND SPECIES OF THE PLANT CLAIMED

Paulownia (genus), species not named

### VARIETY DENOMINATION

Anagenesis Trifolia.

### BACKGROUND OF THE INVENTION

The present invention relates to a new and distinct form of Paulownia tree from the Scrophulariaceae-Figwort Family, botanically known as Paulownia, and referred to by the variety denomination Anagenesis Trifolia. This new Paulownia tree appears to be distinct from other known species in the genus Paulownia. This new Paulownia variety produces a significant increase in fiber volume making it ideal for biomass production.

Paulownia trees are native to mainland China and Southeast Asia. They are easily identified by their large semi-heart shaped leaves and clusters of cascading mauve to white flowers that appear in early spring before the trees go into leaf. Paulownia has been successfully grown in the United States for more than 100 years. However, commercial plantations have only been in existence since the late 1980s. In the Southeast United States, Paulownia often serves as substitute for pine, poplar and other local hardwoods. The Paulownia tree is relatively fast growing and therefore has the ability to regenerate after harvest, making it ideal as a renewable source for timber and fiber for biomass production. The wood is light weight yet strong and useful for plywood, furniture, veneer, oriented strand board, paper making and log homes.

Significant research and development has been carried out to improve the genus and to develop new and distinct varieties that will grow in colder growing zones yet still demonstrate fast growth and drought tolerance. The Paulownia Anagenesis Trifolia is a hybrid resulting from crossing selected offspring of Paulownia Elongata and Paulownia Fortunei with Paulownia Catalpifolia. This variety has the fast growing characteristics and drought tolerance of Paulownia Elongata, the straight growth characteristics of Paulownia Fortunei and the cold tolerance of Paulownia Catalpifolia.

The first act of asexual reproduction of the new form was accomplished in Lenox Ga. The clones of the new form are identical to the original plant in all distinguishing characteristics.

### SUMMARY

The present invention, Paulownia Anagenesis Trifolia, is a new and distinct variety of the genus Paulownia which combines the fast growing characteristics and drought tolerance of Paulownia Elongata, the straight growth characteristics of Paulownia Fortunei and the cold tolerance of Paulownia Catalpifolia. Paulownia Anagenesis Trifolia can mature to a height of 30-35 meters and grow as far north as zone 5A on the USDA plant hardiness zone. This variety can withstand average annual minimum temperature between −15 to −20 F and can be cultivated in areas that have as little as 6-12 inches of rain fall per annum.

The Paulownia Anagenesis Trifolia also distinguishes itself from other Paulownia varieties with its sets of three branches evenly spaced around the tree trunk at approximately 120 degree increments. The additional fiber volume produced as a result of the tripod type branch network increases the fiber production of the tree 20% to 30% annually.

### BRIEF DESCRIPTION OF THE DRAWINGS

The accompanying photographs show specimens of the new variety, with their foliage and flowers in different stages of development.

FIG. 1. A Paulownia Anagenesis Trifolia tree at approximately 1 year in age grown in Lenox Ga.

FIG. 2. A flower the Paulownia Anagenesis Trifolia from 1 year old tree grown in Lenox Ga.

FIG. 3. Branches of a young Paulownia Anagenesis Trifolia 1 year old grown in Lenox Ga.

FIG. 4. Foliage of a young Paulownia Anagenesis Trifolia approximately 6 months old grown in Lenox Ga.

FIG. 5. Typical Paulownia branch net work as is apparent on all other Paulownia Varieties. This photograph is of a 2 year old Paulownia Elongata grown in Lenox Ga.

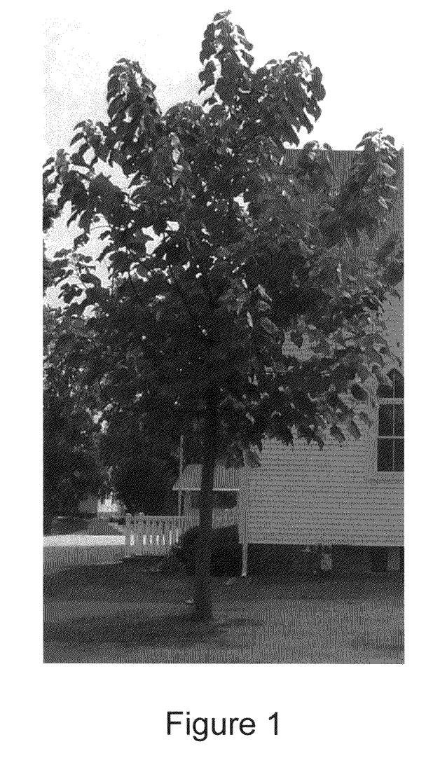

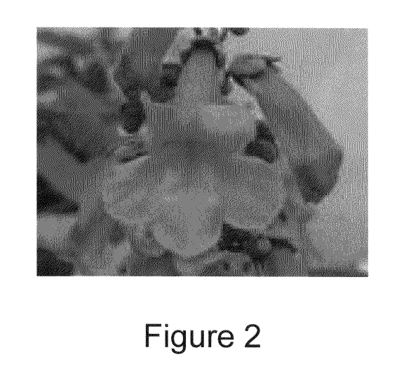

### DETAILED DESCRIPTION OF THE PLANT

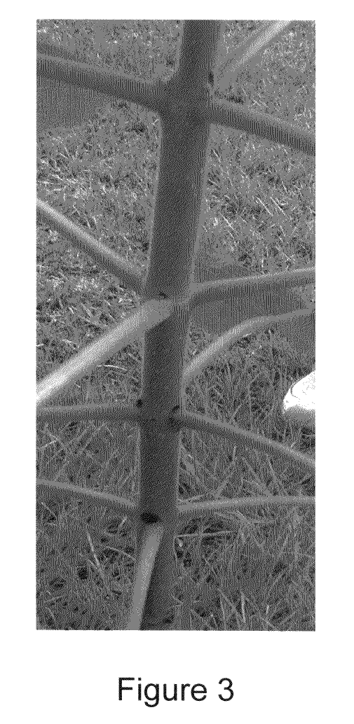

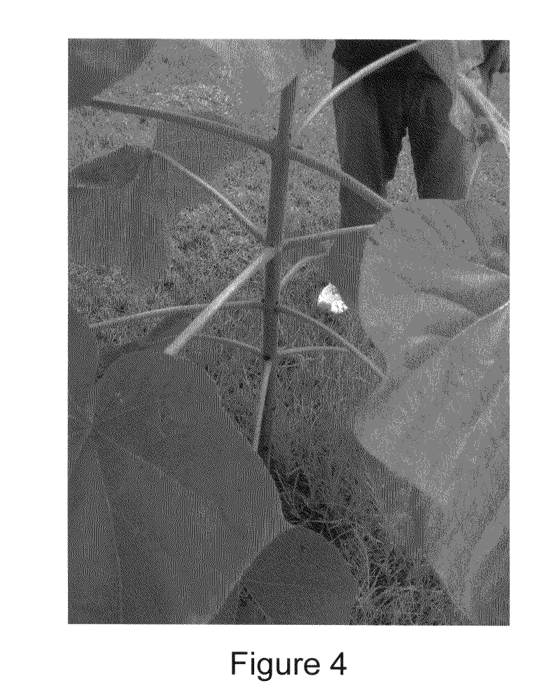

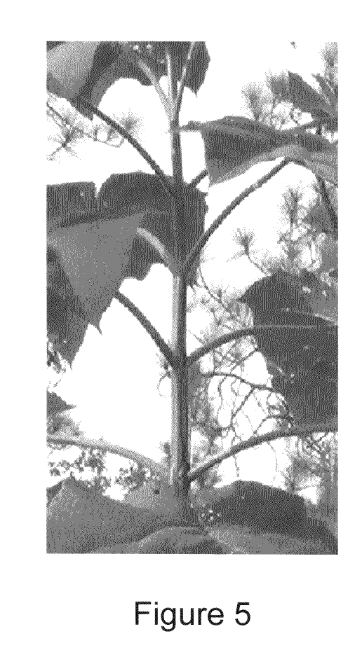

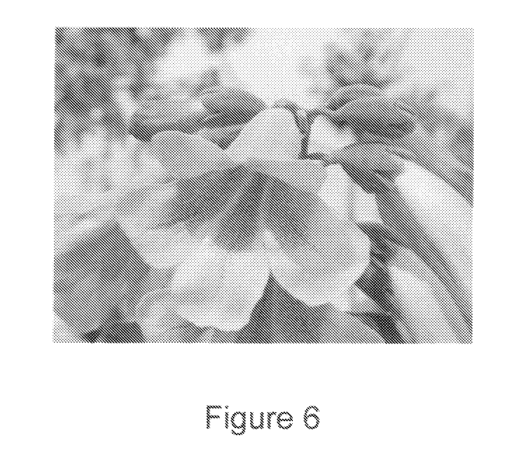

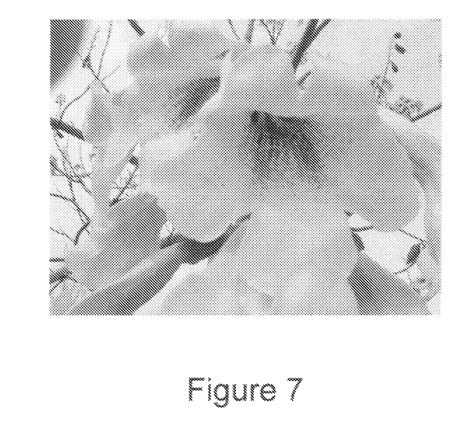

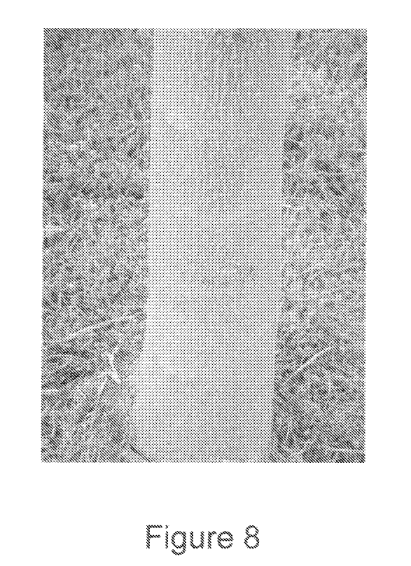

In the following description, color references are made to The Royal Horticultural Society (R.H.S.) Colour Chart, 1995 Edition. Where dimensions, sizes, colors and other characteristics are given, it is to be understood that such characteristics are approximations of averages set forth as accurately as practicable. The descriptions reported herein are from specimens grown at a nursery and field plantings in Lenox Ga.
Botanical classification: Paulownia referred to by the variety denomination Anagenesis Trioflia.
Parentage: The ovary of Paulownia Elongata was fertilized with the pollen of Paulownia Fortunei to produce initial offspring. The ovary of this offspring was fertilized with the pollen of Paulownia Catalpifolia to produce the new and distinct variety of Paulownia.
Propagation: The Paulownia Anagenesis Trifolia has been asexually reproduced in Lennox, Ga. and all clones are identical to the original plant in all distinguishing characteristics. The seeds from Paulownia Anagenesis Trifolia were germinated and grown into seedlings in a greenhouse. Micro-propagation and multiplication of the original seedlings was performed by tissue culture. Rooted tissue cultures were potted in standard potting mix and grown in a greenhouse for 45 days before transplanting to the field.
Plant description: The Paulownia Anagenesis Trifolia is a deciduous hardwood tree that matures to a height of 30-35 meters and has a full umbrella like crown. Mature tree trunk shape is terete and the bark is smooth with a grey color (RHS 197-A to 198-B).
Foliage: Infant leaves reach approximately 20 to 30 inches in diameter, are cordate in shape with visible veins hirsute. Mature leaves are much smaller, approximately 8 to 12 inches in diameter, cordate in shape with visible veins having a smooth margin and a hirsute texture. Color dark green varies from RHS 139-A to 139-C.
Fruits: The bi-valve fruit capsules are ovate in shape and form during the months of April and May. They mature during September and October and are approximately 1.5 inches in length. Hundreds of wing type minute seeds are produced in each capsule.
Flowers: Paulownia Anagenesis Trifolia typically exhibits 6 to 9 cyme inflorescence, approximately 2 inches in length. The flower buds form in autumn and are light green in color. Flowers emerge during March and April displaying a deep mauve (RHS 72-C) to white color. (RHS 155-A). The calyx is fleshy with 5 hirsute elongated lobes and a companulate corolla tube. The ovary is bilocular and the pistil is the same length as the stamens. There are 4 didynamous stamens which are about half as long as the corolla. There are generally two yellow (RHS 3-B) stripes running the length of the corolla. The color of the flowers will vary depending upon soil type, soil fertility and soil pH.
Form and branching: The new variety has a distinct difference in branch characteristics from typical Paulownia trees. Generally, Paulownia tree branches form in pairs along the trunk that grow 180 degrees opposite each other. See FIG. 5. The Paulownia Anagenesis Trifolia however, has sets of three branches evenly spaced around the tree trunk at approximately 120 degree increments. See FIG. 3. The additional fiber volume produced as a result of the tripod type branch network increases the fiber production of the tree 20% to 30% annually.
Tolerance: Paulownia Anagenesis Trifolia is more cold tolerant than most other Paulownia varieties. Most Paulownia trees can be cultivated as far north as zone 6B and can withstand an average annual minimum temperature of 0 to −5 F. However, the Paulownia Anagenesis Trifolia has been successfully grown as far north as USDA plant hardiness zone 5A and can withstand an average annual minimum temperature of −15 to −20 F. This new variety is drought tolerant and can be cultivated in areas that have as little as 6 to 12 inches of rain fall per annum.

1. A new and distinct variety of Paulownia tree as shown and described, characterized by its flower color, fast growing characteristics, cold and drought tolerance, and tri-pod type branch network resulting in increased fiber production.
